(19) United States
(12) Patent Application Publication
(10) Pub. No.: US 2024/0153606 A1
(43) Pub. Date: May 9, 2024

(54) INFORMATION PROVIDING APPARATUS, INFORMATION PROCESSING METHOD, AND RECORDING MEDIUM

(71) Applicant: NEC Corporation, Minato-ku, Tokyo (JP)

(72) Inventors: Yasushi Watanabe, Tokyo (JP); Takuji Ogawa, Tokyo (JP); Yukio Nada, Tokyo (JP); Sayuri Watanabe, Tokyo (JP)

(73) Assignee: NEC Corporation, Minato-ku, Tokyo (JP)

(21) Appl. No.: 18/281,192

(22) PCT Filed: Mar. 15, 2021
(86) PCT No.: PCT/JP2021/010355

§ 371 Date: Sep. 8, 2023

(51) Int. Cl. G16H 20/10; G06V 40/16

(52) U.S. Cl. CPC G16H 20/10; G06V 40/172; G06V 40/178

(57) ABSTRACT

An information processing apparatus provided with a confirmation unit for confirming whether a first face image and a second face image taken of persons receiving online drug administration guidance correspond to a patient-attendant relationship, and an output unit for outputting a confirmation result.

### TECHNICAL FIELD

The present disclosure relates to an information processing apparatus and the like used for online medicine-taking instruction.

### BACKGROUND ART

Before a patient takes a medicine prescribed by a doctor, a medical worker (doctor, pharmacist) is required to instruct the patient to take the medicine in person. In the case of a patient, such as an infant or an elderly person in a welfare facility, who does not have sufficient understanding of medicine-taking instruction, a guardian or a caregiver of the patient together as an attendant receives the medicine-taking instruction to assist the patient with appropriate medication.

As disclosed in PTL 1, online medicine-taking instruction for receiving medicine-taking instruction using a video call function of an information device has started without a patient going to a medical institution (including a pharmacy). The online medicine-taking instruction is an effective method of reducing a burden on a patient such as movement to a medical institution or waiting time.

### CITATION LIST

### Patent Literature

PTL 1: JP 2020-91651 A

### SUMMARY OF INVENTION

### Technical Problem

In order to spread the online medicine-taking instruction, it is necessary to further improve convenience of the system.

### Solution to Problem

An aspect of an information processing apparatus of the present disclosure includes: a confirmation unit configured to confirm whether a first face image and a second face image included in an image obtained by capturing persons who receive online medicine-taking instruction correspond to a relationship between a patient and an attendant; and an output unit configured to output a confirmation result.

An aspect of an information processing method of the present disclosure includes: confirming whether a first face image and a second face image included in an image obtained by capturing an image of a person who receives an online medicine-taking instruction correspond to a relationship between a patient and an attendant; and outputting a confirmation result.

An aspect of a program stored in a recording medium of the present disclosure causes a computer to execute: confirming whether a first face image and a second face image included in an image obtained by capturing an image of a person who receives an online medicine-taking instruction correspond to a relationship between a patient and an attendant; and outputting a confirmation result.

The program may be stored in a non-transitory recording medium which can be readable and writable by a computer.

An object of the present disclosure is to provide a technique for improving convenience of an information processing apparatus in online medicine-taking instruction.

### Advantageous Effects of Invention

According to the information processing apparatus of the present disclosure, it is possible to improve convenience of the information processing apparatus in online medicine-taking instruction.

### BRIEF DESCRIPTION OF DRAWINGS

FIG. 1 is a diagram illustrating an outline of online medicine-taking instruction.

FIG. 2 is a diagram illustrating an outline of online medicine-taking instruction system.

FIG. 3 is a block diagram illustrating an example of a configuration of a user terminal according to the first example embodiment.

FIG. 4 is a diagram illustrating an example of a configuration of a medical DB.

FIG. 5 is a diagram illustrating an example of patient information.

FIG. 6 is a diagram illustrating an example of medical worker information.

FIG. 7 is a diagram illustrating an example of human identification information.

FIG. 8 is a diagram illustrating an example of medicine-taking instruction information.

FIG. 9 is a diagram illustrating an example of payment means information.

FIG. 10 is a block diagram illustrating an example of a configuration of a medical institution terminal according to the first example embodiment.

FIG. 11 is a block diagram illustrating an example of a configuration of an authentication system according to the first example embodiment.

FIG. 12 is a diagram illustrating an example of personal biometric information.

FIG. 13 is a diagram illustrating classification of payers by an age estimation process.

FIG. 14 is a flowchart illustrating an example of an operation of the medical institution terminal according to the first example embodiment.

FIG. 15 is a sequence diagram illustrating an example of a confirmation process.

FIG. 16 is a diagram illustrating an example of screen output of a medical institution terminal.

FIG. 17 is a diagram illustrating another example of the screen output of the medical institution terminal.

FIG. 18 is a sequence diagram illustrating an example of a medicine-taking instruction process.

FIG. 19 is a diagram illustrating another example of the screen output of the medical institution terminal.

FIG. 20 is a sequence diagram illustrating an example of a payment process.

FIG. 21 is a sequence diagram illustrating an example of a medicine-taking notification process.

FIG. 22 is a sequence diagram illustrating an example of a medicine-taking confirmation process.

FIG. 23 is a block diagram illustrating an example of a configuration of a medical institution terminal according to a second example embodiment.

FIG. 24 is a flowchart illustrating an example of an operation of the medical institution terminal according to the second example embodiment.

FIG. 25 is a diagram illustrating an example of a hardware configuration of a computer.

### EXAMPLE EMBODIMENT

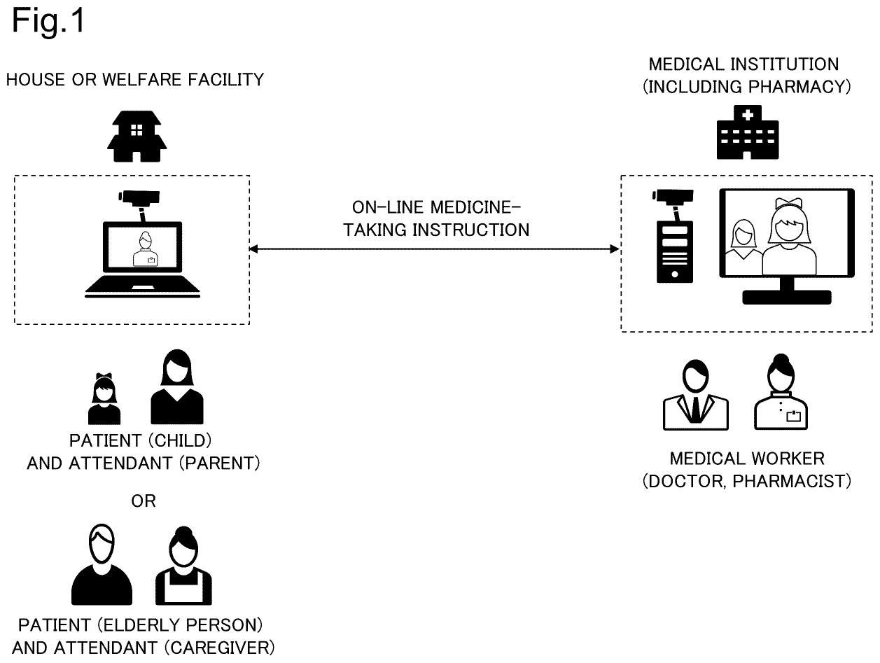

The outline of the online medicine-taking instruction will be described. In the following example, a person who receives the online medicine-taking instruction is a patient and an attendant. FIG. 1 is a diagram illustrating an outline of the online medicine-taking instruction. FIG. 1 illustrates an example in which a patient and an attendant in a house receive online medicine-taking instruction from a medical worker of a medical facility.

The online medicine-taking instruction is achieved, for example, by transmitting and receiving videos and voices to and from each other between a user terminal installed in a patient's residence and a medical institution terminal installed in a medical institution. The user terminal and the medical institution terminal are, for example, a personal computer, a tablet terminal, a smartphone, and the like. In the online medicine-taking instruction, a pharmacist explains how to take a medicine prescribed by a doctor to a patient and an attendant via a personal computer. The attendant is, for example, a person who assists a patient to take medicine by receiving online medicine-taking instruction. The place where the online medicine-taking instruction is received may be, for example, a welfare facility other than the patient's residence. In this case, the patient is an elderly person, and the attendant is a caregiver.

### First Example Embodiment

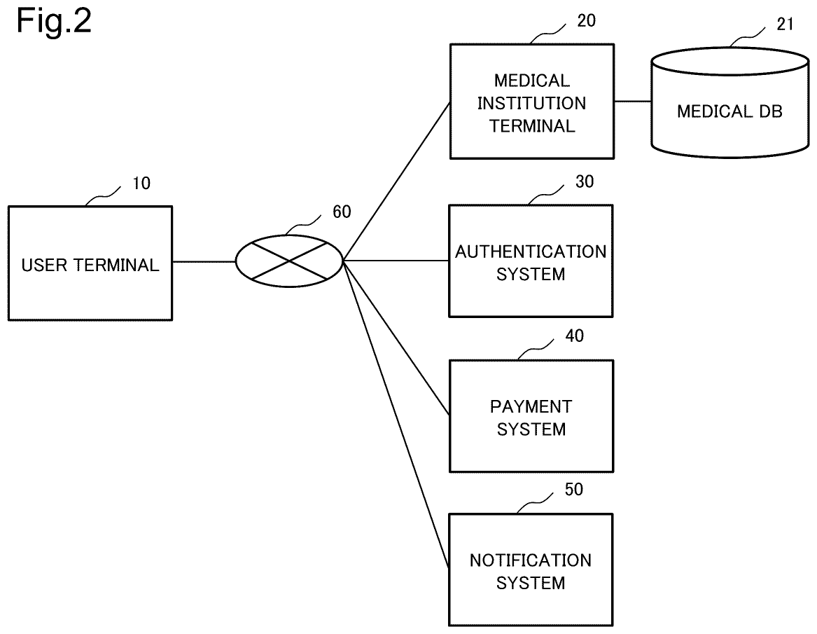

An online medicine-taking instruction system of a first example embodiment will be described with reference to the drawings. FIG. 2 is a diagram illustrating an outline of the online medicine-taking instruction system. In the online medicine-taking instruction system, a user terminal 10 and a medical institution terminal 20 are communicably connected via a network 60. The medical institution terminal 20 is communicably connected to an authentication system 30, a payment system 40, and a notification system 50 via the network 60.

The medical institution terminal 20 is communicably connected to a medical DB 21. The medical institution terminal 20 may be connected to the medical DB 21 via the network 60 as long as secure communication is possible.

[User Terminal]

The user terminal 10 will be described. The user terminal 10 is a terminal used by a person who receives the online medicine-taking instruction. The person who receives the online medicine-taking instruction is, for example, a patient and an attendant thereof. An attendant who assists the patient in taking medicine may receive online medicine-taking instruction instead of the patient.

The user terminal 10 is, for example, a personal computer, a tablet, a smartphone, or the like. The user terminal 10 is not limited to the terminal owned by the user. For example, it may be an information communication terminal held by a welfare facility. The user terminal 10 communicates with the medical institution terminal 20, and transmits and receives a video and a voice for online medicine-taking instruction.

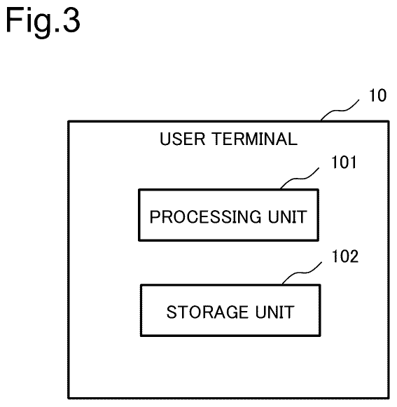

FIG. 3 is a block diagram illustrating an example of a configuration of the user terminal 10 according to the first example embodiment. The user terminal 10 includes a processing unit 101 and a storage unit 102. The user terminal 10 achieves various functions by executing a processing program stored in the storage unit 102. The user terminal 10 may include a camera, a microphone, a monitor, a speaker, and a communication unit (not illustrated). The camera, the microphone, the monitor, and the speaker may be devices built in the user terminal 10 or may be external devices. The user terminal 10 acquires images and voices of the patient and the attendant who receive the online medicine-taking instruction via the camera and the microphone.

The processing unit 101 displays and outputs the acquired video on the monitor, and outputs the acquired audio from the speaker. The processing unit 101 transmits an image (captured data) obtained by photographing the patient and the attendant and a voice to the medical institution terminal 20.

The processing unit 101 may transmit the verification information to the medical institution terminal 20. The verification information is information for specifying an individual by an identifier attached to the identity verification. The identity verification is, for example, a health insurance card, a personal number card (My Number Card), a driver's license, or a passport. The identity verification is not limited to a health insurance card or the like as long as a unique identifier is given to an individual. For example, the processing unit 101 may extract the verification information from the IC chip of the personal number card and transmit the verification information to the medical institution terminal 20, or may transmit the captured images of the front and back of the personal number card as the verification information.

At the time of online medicine-taking instruction, the processing unit 101 may store information of medicine-taking instruction such as a prescription of medicine transmitted from the medical institution terminal 20 in a storage unit 103.

The processing unit 101 may transmit a medicine-taking report indicating that a prescribed medicine has been taken to the medical institution terminal 20. The medicine-taking report is a report indicating the medicine-taking status of the patient. The medicine-taking status may be information indicating whether the medicine can be taken according to the medicine-taking instruction. The medicine-taking report may include a medicine-taking time in addition to the medicine taken by the patient. The medicine-taking report may include a video of the patient taking the medicine.

The storage unit 103 stores information on medicine-taking instruction and an application for online medicine-taking instruction.

[Medical DB]

In the medical DB, information regarding medical care and treatment of a patient is recorded. The medical DB may include information on prescriptions for each patient.

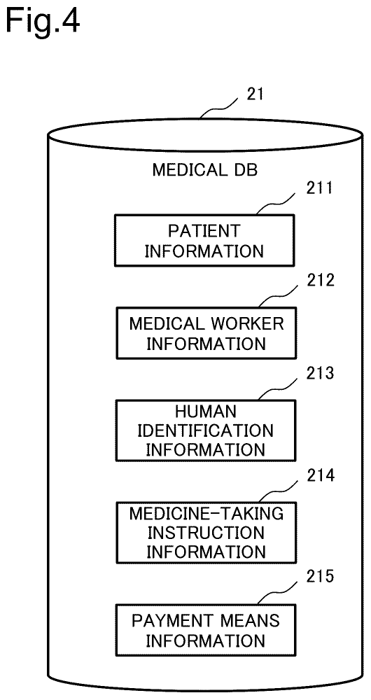

FIG. 4 is a diagram illustrating an example of a configuration of the medical DB 21. The medical DB 21 stores patient information 211, medical worker information 212, human identification information 213, medicine-taking instruction information 214, and payment means information 215.

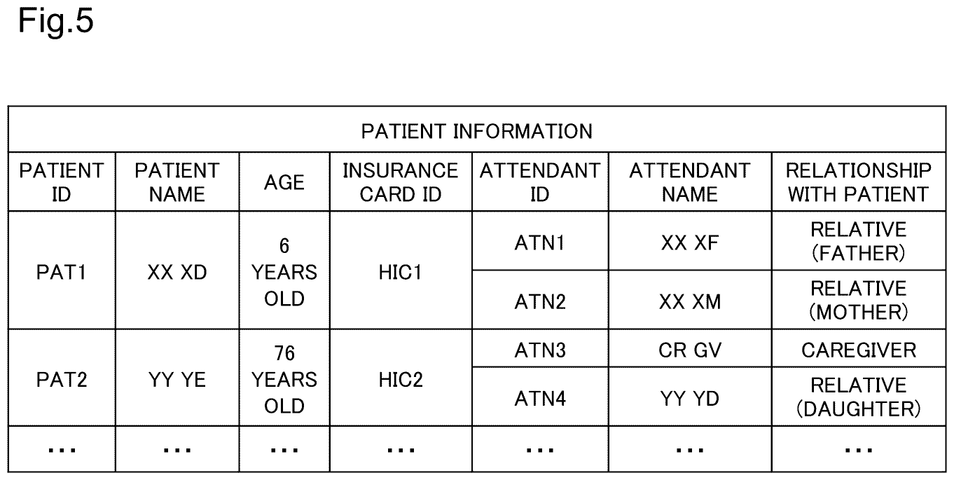

FIG. 5 is a diagram illustrating an example of the patient information 211. The patient information 211 is information related to the patient. The patient information 211 includes, for example, items of a patient identifier (ID), a patient name, an age, and an insurance card ID. The patient information 211 illustrated in FIG. 5 includes information on an attendant of the patient (also referred to as attendant information). The attendant information is, for example, an attendant ID, an attendant name, and a relationship with a patient. The relationship with the patient is, for example, relationship information such as a father and a daughter, or information of a welfare-related person such as a caregiver. The patient information 211 may include items other than the above items. For example, disease information associated with the patient ID may be included. The patient information 211 may be stored in a storage unit (not illustrated) of the medical institution terminal 20.

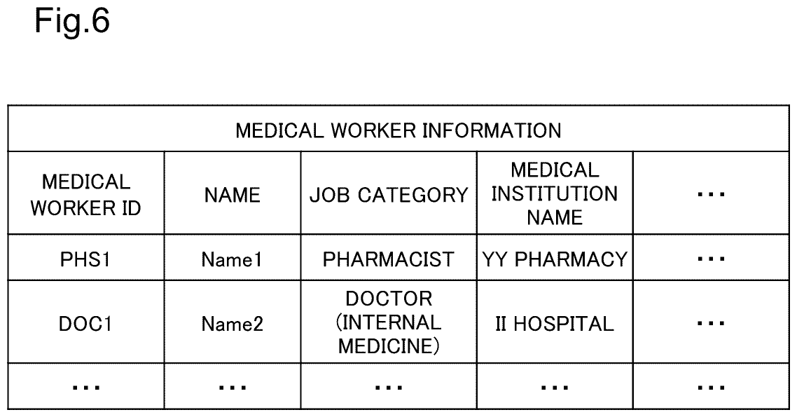

FIG. 6 is a diagram illustrating an example of the medical worker information 212. The medical worker information 212 is information on a medical worker. The medical worker information 212 includes items of a medical worker ID, a name, a job category, and a medical institution name. The job category indicates a job category of a medical worker such as a pharmacist or a doctor. The medical institution name indicates a place of employment of a medical worker such as a YY pharmacy or a II hospital. The medical worker information 212 may include items other than the above items. The medical worker information 212 may be stored in a storage unit (not illustrated) of the medical institution terminal 20.

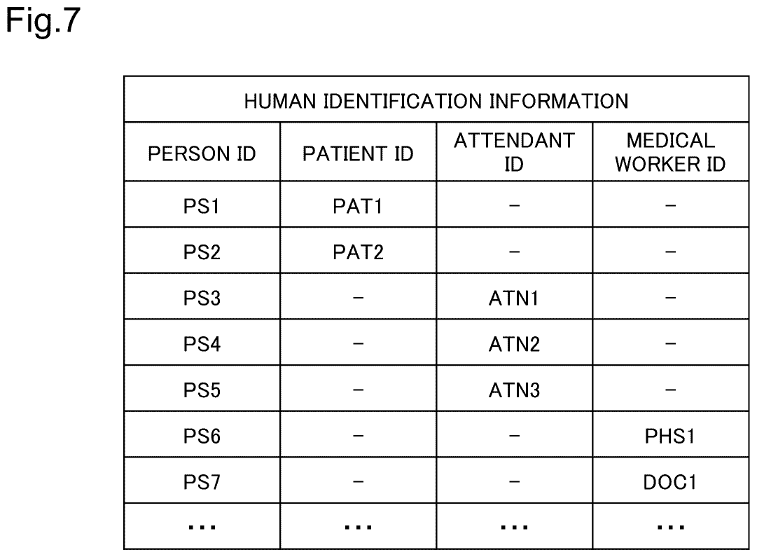

FIG. 7 is a diagram illustrating an example of the human identification information 213. The human identification information 213 is information in which a person ID for identifying an individual is associated with a patient ID, an attendant ID, and a medical worker ID included in the medical DB 21. The human identification information 213 may include items other than the above items.

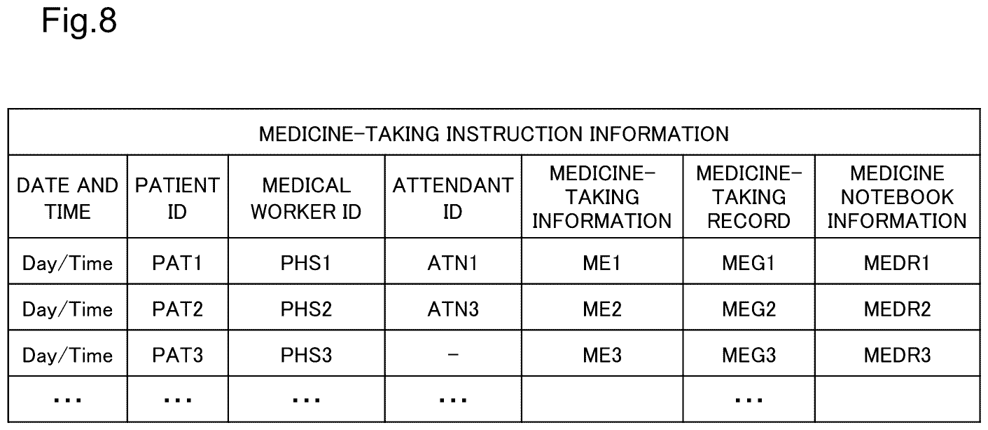

FIG. 8 is a diagram illustrating an example of the medicine-taking instruction information 214. The medicine-taking instruction information 214 illustrated in FIG. 8 includes items of date and time, a patient ID, a medical worker ID, an attendant ID, medicine-taking information, a medicine-taking instruction record, and medicine notebook information. The date and time indicates a date and time when the medicine-taking instruction is given to the patient. The medicine-taking information includes a medicine prescribed to a patient and information on the prescription. The medicine-taking instruction record includes the content of the medicine-taking instruction to the patient. The medicine-taking instruction record may include recorded data of online medicine-taking instruction. The medicine notebook information is information such as a patient's medicine-taking history, previous diseases, and allergies.

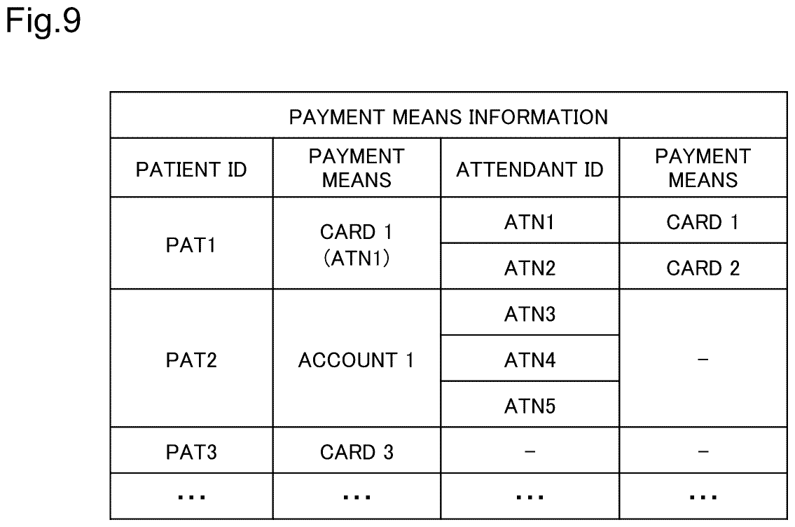

FIG. 9 is a diagram illustrating an example of the payment means information 215. The payment means information 215 is information in which the patient ID and the payment means are associated with each other. The payment means information 215 illustrated in FIG. 9 is information in which the patient and the payment means of the attendant are associated with each other.

The payment means is, for example, a credit card, account transfer, or the like. The payment means may be a two-dimensional code used in mobile payment or an IC card in electronic money payment. For example, in the case of a credit card, credit card information such as a credit card name, a credit card number, an expiration date, and a registrant name is used for payment. In the case of account transfer, account information such as a financial institution name, a branch name, an account number, and an account name is used.

In the payment means information 215 of FIG. 9, the patient ID “PAT1” (child) is associated with the “Card 1 (ATN1)” of the parent who is an attendant, and the credit cards “card 1” and “card 2” are associated with the “ATN1” (father) and “ATN2” (mother) of the attendant IDs. The account transfer “account 1” is associated with the patient ID “PAT2” (elderly person), and the payment means is not associated with each of the attendants “ATN3” (caregiver), “ATN4” (caregiver), and “ATN5” (daughter).

For example, the medical institution terminal 20 refers to the payment means information 215 of the medical DB 21, reads out the payment means associated with the patient to whom the online medicine-taking instruction is performed, and uses the same for the payment process for the online medicine-taking instruction.

[Medical Institution Terminal]

The medical institution terminal 20 will be described. The medical institution terminal 20 is a terminal used by a person who performs online medicine-taking instruction. The person who performs the online medicine-taking instruction is, for example, a doctor or a pharmacist. The medical institution terminal 20 is, for example, a personal computer, a server, or the like. The medical institution terminal 20 communicates with the user terminal 10, and transmits and receives a video and a voice for online medicine-taking instruction. The medical institution terminal 20 may transmit information regarding medicine-taking instruction to the user terminal 10.

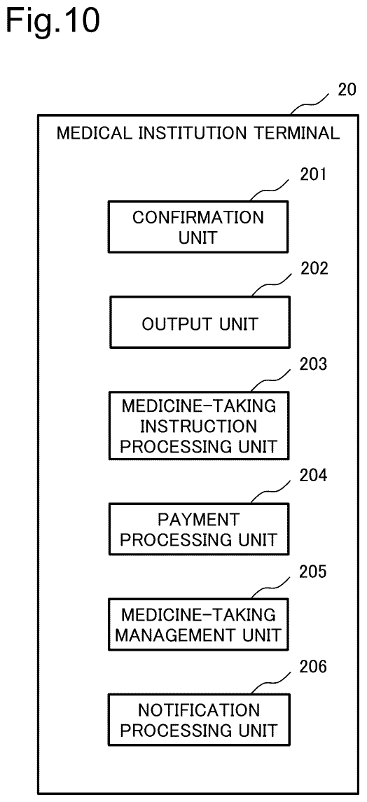

FIG. 10 is a block diagram illustrating an example of a configuration of the medical institution terminal 20 according to the first example embodiment. The medical institution terminal 20 illustrated in FIG. 10 includes a confirmation unit 201, an output unit 202, a medicine-taking instruction processing unit 203, a payment processing unit 204, a medicine-taking management unit 205, and a notification processing unit 206. The medical institution terminal is also referred to as an information processing apparatus.

The medical institution terminal 20 may include a camera, a microphone, a monitor, a speaker, and a communication unit (not illustrated). Each of the camera, the microphone, the monitor, and the speaker may be a device built in the medical institution terminal 20 or an external device.

The medical institution terminal 20 includes a processor and a memory, and the processor may execute a processing program stored in the memory to implement various functions including the functions of the confirmation unit 201, the output unit 202, the medicine-taking instruction processing unit 203, the payment processing unit 204, the medicine-taking management unit 205, and the notification processing unit 206. At least one of the medicine-taking instruction processing unit 203, the payment processing unit 204, the medicine-taking management unit 205, and the notification processing unit 206 may be omitted from the configuration of the medical institution terminal 20.

When the medical institution terminal 20 receives the face image of the person who receives the online medicine-taking instruction transmitted from the user terminal 10, the confirmation unit 201 confirms whether the person of the acquired face image is the patient who receives the online medicine-taking instruction and the attendant thereof. Specifically, the confirmation unit 201 confirms whether the acquired two face images (first face image, second face image) correspond to the relationship between the patient and the attendant. For this confirmation, the confirmation unit 201 executes an identity confirmation process and a relationship confirmation process. In a case where there is a single person who receives the medicine-taking instruction, the confirmation unit 201 may not execute the relationship confirmation process.

In the identity confirmation process, the confirmation unit 201 transmits the face image to the authentication system 30 and collates the face image with the face information registered in the authentication system 30. In a case where a plurality of face images are included in one image, the confirmation unit 201 extracts a face image for each face and transmits the face image to the authentication system 30. For example, the face image may be transmitted to the authentication system along with the request number. The process of extracting the face image for each face may be executed by the authentication system 30. In this case, the confirmation unit 201 transmits one image including a plurality of face images to the authentication system 30.

When the face information of the face image matches the face information of the authentication system 30, the authentication system 30 transmits the person ID relevant to the registered face information to the medical institution terminal 20 as the collation result. When the face information does not match the face information of the authentication system 30, the authentication system 30 transmits a collation error to the medical institution terminal 20 as a collation result.

When receiving the person ID of the collation result from the authentication system 30, the confirmation unit 201 refers to the human identification information 213 of the medical DB 21 and extracts a patient ID or an attendant ID that matches the person ID of the collation result.

When receiving the collation error from the authentication system 30, the confirmation unit 201 passes the collation error to the output unit 202 as the confirmation result without executing the relationship confirmation process. The output unit 202 may display and output the collation error as error information.

Next, in the relationship confirmation process, the confirmation unit 201 confirms whether the extracted patient ID and attendant ID correspond to the relationship between the patient ID and the attendant ID registered in the patient information 211 of the medical DB 21.

In a case where the relationship corresponds to the relationship between the patient and the attendant registered in the patient information 211, the confirmation unit 201 sends the confirmation result to the output unit 202 and the medicine-taking instruction processing unit 203. The confirmation result may include the patient ID of the patient whose identity verification has been completed and the attendant ID of the attendant. The confirmation result may include a patient name, an attendant name, or a relationship with a patient.

On the other hand, in a case where the relationship does not correspond to the relationship between the patient and the attendant, the confirmation unit 201 transmits the error information to the output unit 202 as a confirmation result.

In a case where the accounting of the online medicine-taking instruction is processed by face payment, the confirmation unit 201 may transmit the collation result obtained by the identity confirmation process of the patient and the attendant to the payment processing unit 204.

In the above description, the confirmation unit 201 confirms the identity of the person who receives the medicine-taking instruction, but may confirm the identity of a doctor or a pharmacist who performs the medicine-taking instruction. In this case, the confirmation unit 201 collates the face image of the person performing the medicine-taking instruction with the authentication system 30 using the medical institution terminal 20, and refers to the collation result and the medical worker information 212 to confirm that the person is a doctor or a pharmacist.

The output unit 202 outputs the confirmation result. For example, the output unit 202 may output information indicating that the face of the person receiving the medicine-taking instruction or the periphery thereof displayed on the monitor screen of the medical institution terminal 20 has been identified. For example, the information indicating that the identity verification is obtained can be output while superimposed on the face image. The output unit 202 may display and output the person who receives the medicine-taking instruction, for example, the patient name and the attendant name on the monitor screen. The output unit 202 may transmit the image of the monitor screen of the medical institution terminal 20 to the user terminal 10. The output unit 202 may output error information indicating that there is no relationship between the persons in the first face image and the second face image.

The output unit 202 may output information included in the patient information 211, the medical worker information 212, the medicine-taking instruction information 214, and the payment means information 215. The output destination is a monitor of the medical institution terminal 20. The output unit 202 may output the image obtained by capturing the monitor screen to the user terminal 10.

The output unit 202 may receive the patient name or the attendant name who is the payer of the accounting from the payment processing unit 204 to be described later, and display and output the payment confirmation information. The payment confirmation information is, for example, “Is the payer of the accounting Mr./Ms. ∘∘?”. The output destination is the monitor screen of the medical institution terminal 20 or the user terminal 10.

After completion of the identity confirmation and the relationship confirmation of the person who receives the medicine-taking instruction, online medicine-taking instruction to the patient and the attendant is started. The medicine-taking instruction processing unit 203 generates a record of the medicine-taking instruction information 214 illustrated in FIG. 8 using the patient ID and the attendant ID sent from the confirmation unit 201, and adds the record to the medicine-taking instruction information 214 of the medical DB.

The medicine-taking instruction processing unit 203 registers a medicine prescribed by a doctor to a patient and information (prescription information) regarding the prescription to the medicine-taking information.

In the online medicine-taking instruction, a pharmacist and a doctor who give instruction on medicine-taking instruction listen to symptoms from a patient and an attendant, and then explain an effect of a prescribed medicine, a method of taking the medicine, a possible side effect, a response to forgetting to take the medicine, and a storage method. The pharmacist and the doctor confirm and answer questions from the patient and the attendant.

After the medicine-taking instruction, the medicine-taking instruction processing unit 203 registers the medicine history of the patient in the medicine-taking instruction record of the medicine-taking instruction information 214. As the medicine history, a format of an SOAP format may be used.

The medicine history includes subjective information spoken by a patient or an attendant, objective information such as a test result, pharmaceutical consideration, schedule of future care, and the like.

The medicine-taking instruction record may include recorded data from the start to the end of the online medicine-taking instruction. The recorded data of the online medicine-taking instruction may be browsed at a later date in response to a request from the patient or the attendant. However, it is preferable to set the condition that the identity confirmation process is performed on the patient and the attendant and the confirmation is completed by the relationship confirmation process. Identity confirmation and relationship confirmation may be performed by other than face authentication, such as password authentication using a password registered in advance.

The medicine-taking instruction processing unit 203 registers the medicine-taking information in the medicine notebook information of the patient after ending the medicine-taking instruction.

The payment processing unit 204 refers to the payment means information 215 in the medical DB 21 and acquires information on the payment means associated with the patient or the attendant. The payment processing unit 204 transmits a payment confirmation request for confirming a payment means associated with the patient or the attendant to the patient or the attendant who is the payer in the accounting of the online medicine-taking instruction. For example, the payment processing unit 204 transmits the payment confirmation to the user terminal 10 via the output unit 202. In a case where the payment confirmation result from the user terminal 10 is payment approval, the payment processing unit 204 transmits information on the payment means associated with the patient or the attendant to the payment system 40, and executes the payment process.

The payment processing unit 204 confirms the payment confirmation result from the user terminal 10, and when the payment confirmation result indicates payment approval, transmits information of the payment means to the payment system 40 to request payment. The payment system 40 makes a payment with the transmitted information on the payment means, and transmits the payment result to the medical institution terminal 20.

When the payment result received from the payment system 40 indicates payment completion, the payment processing unit 204 completes the payment process. The payment processing unit 204 may transmit the information on the payment completion to the user terminal 10 via the communication unit.

In a case where the identification of the patient and the attendant is confirmed using the face image, the payment processing unit 204 may determine the payer of the accounting for the online medicine-taking instruction using the result of an age estimation process based on the face image of the patient and the attendant. For example, as a result of the age estimation process, the payment processing unit 204 specifies a patient with a face image with an estimated age higher or an attendant as the payer. The payment processing unit 204 may transmit a payment confirmation request for confirming a payment means associated with the identified patient or the attendant. When the processing unit 101 of the user terminal 10 outputs the payment confirmation display, the patient or the attendant can confirm and register the payer and the payment means. The output image by the processing unit 101 is, for example, “Is the payer Mr./Ms. ∘∘?”.

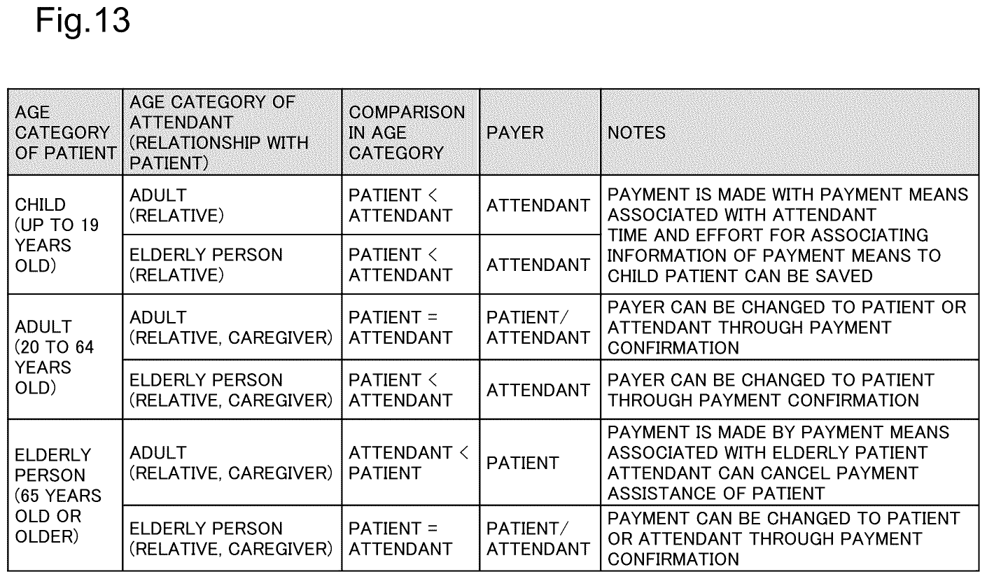

FIG. 13 is a diagram illustrating classification of payers by an age estimation process. In the example of FIG. 13, the age of the patient and the attendant for which the age estimation process has been performed is divided into a child (up to 19 years old), an adult (20 to 64 years old), and an elderly person (65 years old or older). The age category is an example and is not limited thereto.

As a result of the age estimation process, in a case where the patient is a child and the attendant is an adult or older age group, a relative who is an elderly attendant is identified as a payer, and payment is made by a payment means associated with the attendant. In such an age classification case, it is possible to save time and effort for registering the information on the payment means in association with the child patient of the payment means information 215. As a result of the age estimation process, in a case where the patient is an elderly person and the attendant is an adult age category, the payment process can proceed by the payment means associated with the patient who is the elderly person.

In a case where the patient and the attendant have the same age classification, the payer can be changed to one payer by confirming whether the payer is the patient or the attendant through payment confirmation. In a case where the attendant is an adult patient and the age category of the elderly person, the attendant is identified as the payer, but the payment can be changed to the payment of the patient by the payment confirmation.

For example, the medical institution terminal 20 refers to the payment means information 215 of the medical DB 21, reads out the information of the payment means associated with the patient who has performed the online medicine-taking instruction, and uses the information for the payment process for the online medicine-taking instruction.

The medicine-taking management unit 205 performs medicine-taking management for the patient who has received the online medicine-taking instruction. The medicine-taking management includes a medicine-taking notification process and a medicine-taking confirmation process.

By the medicine-taking notification process, the timing at which the patient takes the medicine is notified to the patient or the attendant. For example, the medicine-taking management unit 205 refers to the medicine-taking information and the medicine-taking instruction record in the medical DB 21, acquires a time zone when the patient takes the medicine, and generates the medicine-taking notification. The time zone of taking the medicine is, for example, morning, daytime, or night. The medicine-taking notification may include the patient name, the prescribed medicine, and the dosage thereof in addition to the time zone.

The medicine-taking status of the patient is confirmed by the medicine-taking confirmation process. For example, the medicine-taking management unit 205 sends a medicine-taking confirmation notification to the user terminal 10, accepts a medicine-taking report from the user terminal 10, and confirms the medicine-taking status of the patient. The medicine-taking management unit 205 may register the confirmed medicine-taking status of the patient in the medicine-taking instruction record of the medical DB 21. The medicine-taking management unit 205 refers to, for example, the medicine-taking information and the medicine-taking instruction record in the medical DB 21, and generates a medicine-taking confirmation notification of the patient.

The medicine-taking management unit 205 may generate a remaining medicine notification of a medicine prescribed with reference to the prescription instruction record of the patient updated by the medicine-taking report of the patient. The remaining medicine notification may include a date when the number of remaining medicines becomes zero at the shortest. The remaining medicine notification may include information related to a reservation of the next online medicine-taking instruction. The information regarding the reservation may be, for example, “You will have no medicine on ∘∘ days later. If you need to add information, please contact the ΔΔ doctor's office and contact the next online medicine-taking instruction. Next reservation contact: https://∘∘.jp/OnlineFukuyaku/”. The information regarding the reservation may include, for example, a schedule in which online medicine-taking instruction can be reserved. The information regarding the online medicine-taking instruction reservation may be notified as a medicine-taking instruction reservation notification separately from the remaining medicine notification. The target of the reservation may be online medical care, visit medical care, or outpatient medical care in addition to the online medicine-taking instruction.

The medicine-taking management unit 205 passes the medicine-taking notification, the medicine-taking confirmation notification, or the remaining medicine notification to the notification processing unit 206.

The notification processing unit 206 generates a notification instruction, and transmits the generated notification instruction to the notification system 50. The notification instruction includes notification contents to the patient who has received the online medicine-taking instruction or the attendant. The notification content is, for example, a medicine-taking notification, a medicine-taking confirmation notification, or a remaining medicine notification.

[Authentication System]

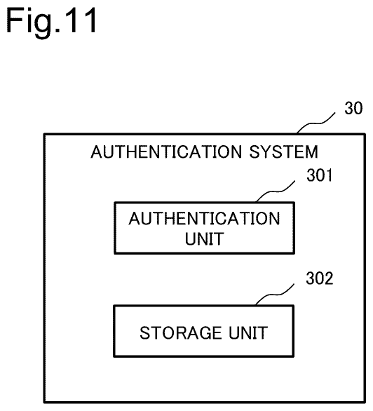

FIG. 11 is a block diagram illustrating an example of a configuration of the authentication system 30. The authentication system 30 illustrated in FIG. 11 includes an authentication unit 301 that performs authentication using biometric information and a storage unit 302 that stores personal biometric information. The biometric information is, for example, face information included in a face image.

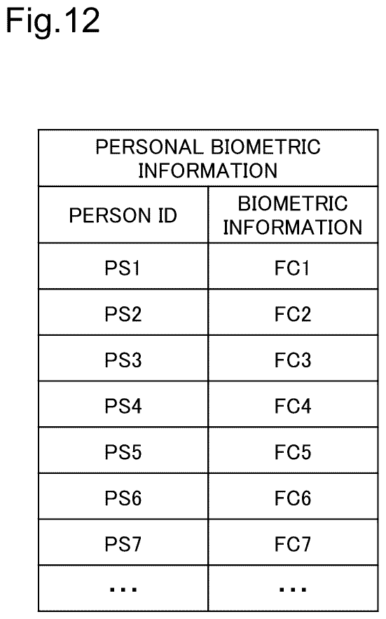

FIG. 12 is a diagram illustrating an example of personal biometric information. The personal biometric information illustrated in FIG. 12 includes, for example, items of a person ID for identifying an individual and biometric information of the individual. The biometric information may include face information, fingerprint information, and palm print information. The face information is, for example, a feature amount of the face.

The authentication unit 301 extracts the face information from the received face image and collates the face information with the personal biometric information (face information) in the storage unit 302. When the authentication unit 401 detects a plurality of faces in the received face image, the authentication unit 401 collates the faces one by one.

In a case where the face information of the received face image matches the face information in the storage unit 302, the authentication system 30 transmits the person ID relevant to the registered face information to the medical institution terminal 20 as the collation result. When the face information does not match the face information of the authentication system 30, the authentication system 30 transmits a collation error as a collation result to the medical institution terminal 20.

Although the authentication system 30 has been described as a device external to the medical institution terminal 20, the medical institution terminal 20 may include the function of the authentication system 30.

[Payment System]

The payment system 40 illustrated in FIG. 2 performs a payment process such as credit card payment or account transfer payment. The payment system 40 may be a payment proxy system that collectively processes payment by various payment means. The connection method with the payment system 40 may be, for example, any of a link type, a token type, a data transmission type, and a mail link type, and may be another type.

The payment system 40 accepts a payment request from the medical institution terminal 20, performs credit collation and sales processing, and returns a result.

At the time of the payment process, the payment system 40 receives, for example, a credit card number, an expiration date, and the like of the payment means (card 1) associated with the patient ID (PAT1) from the medical institution terminal 20. The payment system 40 is online connected to a server such as a card company, checks the validity of the card 1, and performs sales processing. The payment system 40 transmits the payment result to the medical institution terminal 20 and records the payment result in the transaction log.

Although the payment system 40 has been described as a device external to the medical institution terminal 20, the medical institution terminal 20 may include the function of the payment system 40.

[Notification System]

The notification system 50 illustrated in FIG. 2 receives the notification instruction transmitted by the medical institution terminal 20, and notifies the notification contents to the patient and the attendant based on the notification instruction. The notification to the patient and the attendant may be received by the user terminal 10. The notification may be received by a user terminal different from the user terminal 10 used in the online medicine-taking instruction. For example, online medicine-taking instruction may be performed on a personal computer, and notification may be received by a smartphone. The notification system 50 is, for example, a server that provides an email system for managing an email address and an SNS (SOCIAL NETWORKING SERVICE).

Although the notification system 50 has been described as a device external to the medical institution terminal 20, the medical institution terminal 20 may include the function of the notification system 50.

[Operation]

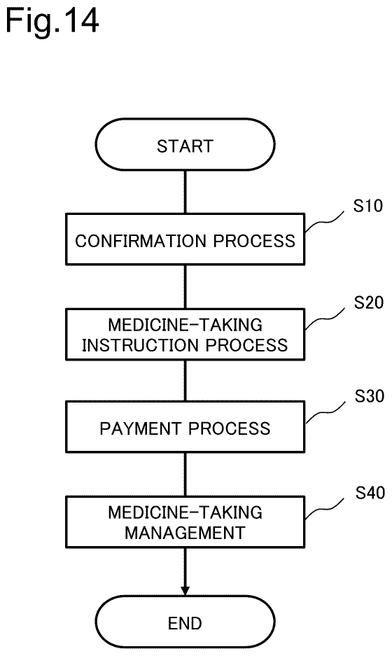

An operation of the medical institution terminal according to the first example embodiment will be described with reference to the drawings. FIG. 14 is a flowchart illustrating an example of the operation of the medical institution terminal 20. The medical institution terminal 20 executes a confirmation process (step S10), a medicine-taking instruction process (step S20), a payment process (step S30), and medicine-taking management (step S40).

Before starting the confirmation process, the user terminal 10 and the medical institution terminal 20 are communicably connected to each other and can transmit and receive various types of information. The confirmation process (step S10) of the medical institution terminal 20 illustrated in FIG. 14 will be described. The confirmation process includes an identity confirmation process and relationship confirmation process. The confirmation process is executed before the start of the medicine-taking instruction.

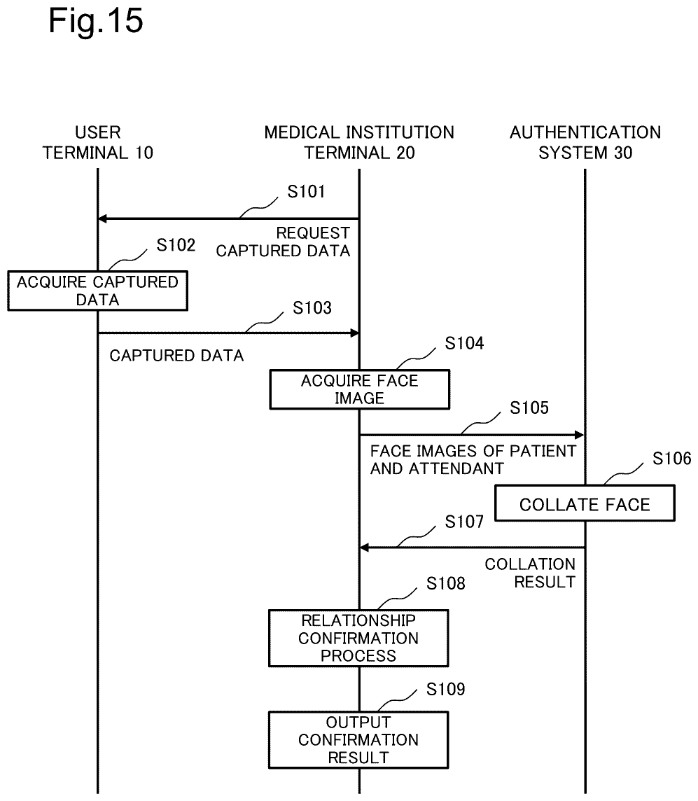

FIG. 15 is a sequence diagram illustrating an example of the confirmation process. The confirmation unit 201 of the medical institution terminal 20 requests the user terminal 10 for the captured data for the identity confirmation process (step S101). The processing unit 11 of the user terminal 10 acquires captured data obtained by photographing the appearance of the patient and the attendant (step S102). The captured data is captured by a camera connected to or built in the user terminal 10. The user terminal 10 transmits the captured data including the face of the patient who receives the online medicine-taking instruction and the attendant to the medical institution terminal 20 (step S103).

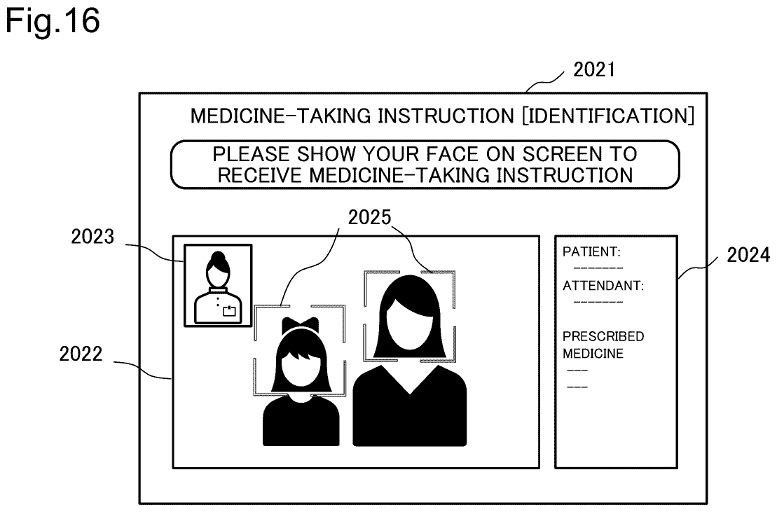

When the medical institution terminal 20 receives the captured data of the patient and the attendant, the output unit 202 outputs the captured data to the monitor screen of the medical institution terminal 20. FIG. 16 is a diagram illustrating a screen output of the medical institution terminal 20. A screen display 2021 illustrated in FIG. 16 includes an image display 2022 of the captured data of a patient and an attendant, an image display 2023 of the captured data of a pharmacist acquired by the medical institution terminal 20, and a display 2024 of information related to medicine-taking instruction.

A mark 2025 is superimposed and displayed on the image display 2022 of the captured data. The mark 2025 indicates that the face image is included in the captured data. The face of the captured data is recognized by a known face recognition technology. The confirmation unit 201 determines whether the face information can be extracted from the recognized face image, and the output unit 202 changes the color of the mark according to the determination. The doctor or pharmacist looks at the image display 2022 of the captured data and the display of the mark 2025, and guides the patient and the attendant so that the face images of the patient and the attendant can be acquired from the captured data.

Returning to FIG. 15, the confirmation unit 201 acquires the face image (first face image) of the patient and the face image (second face image) of the attendant (step S104), and the medical institution terminal transmits the face images of the patient and the attendant to the authentication system 30 (step S105).

The authentication system 30 extracts face information for each of the received face image (first face image) of the patient and the received face image (second face image) of the attendant, and collates the face information with face information registered in advance in the authentication system 30 (step S106).

The authentication system 30 transmits the collation result to the medical institution terminal 20 (step S107). In a case where the face information of the patient and the face information of the attendant coincide, the collation result including the person ID relevant to the face information registered in the authentication system 30 is transmitted. On the other hand, when the face information does not match, the collation result including the collation error is transmitted.

When the medical institution terminal 20 receives the person ID as the collation result, the confirmation unit 201 executes a relationship confirmation process (step S108). The confirmation unit 201 refers to the human identification information 213 of the medical DB 21, and extracts a patient ID matching the person ID of the collation result or an attendant ID. The confirmation unit 201 confirms whether the extracted patient ID and attendant ID correspond to a relationship between the patient ID and the attendant ID registered in the patient information 211 of the medical DB 21.

As a result of the confirmation, in a case where the relationship corresponds to the relationship between the patient and the attendant, the confirmation unit 201 sends the confirmation result to the output unit 202 and the medicine-taking instruction processing unit 203. The confirmation result may include the patient ID of the patient whose identity verification has been completed and the attendant ID of the attendant. The confirmation result may include a patient name, an attendant name, or a relationship with a patient.

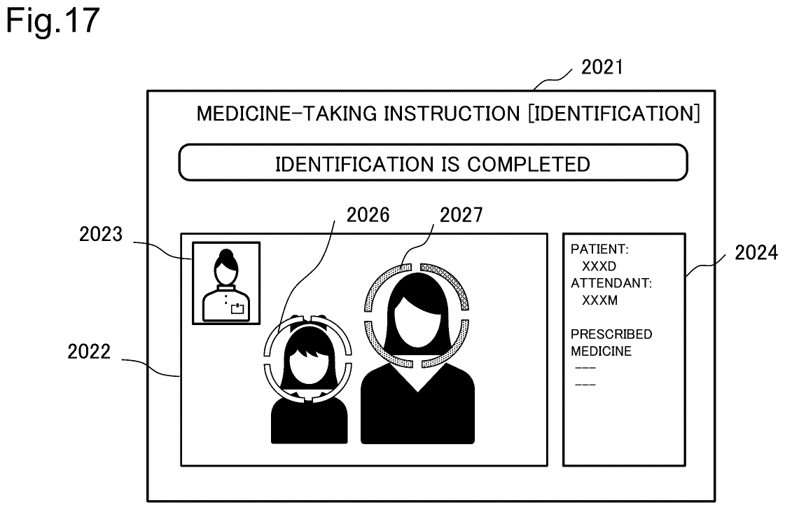

The output unit 202 of the medical institution terminal 20 outputs the confirmation result (step S109). FIG. 17 is a diagram illustrating a screen output of the medical institution terminal 20. The screen display 2021 illustrated in FIG. 17 illustrates a display when the confirmation process is completed. The marks 2026 and 2027 indicate that the patient and the attendant of the captured data correspond to the relationship between the patient and the attendant registered in advance. By associating the position of the face image in the captured data with the confirmation result of the face image, the color, the shape, and the like of the marks 2026 and 2027 may be changed in the image display 2022 so that the doctor and the pharmacist can identify which one of the patient and the attendant is. Further, after completion of the confirmation process, the output unit 202 displays and outputs the names of the patient and the attendant on the display 2024.

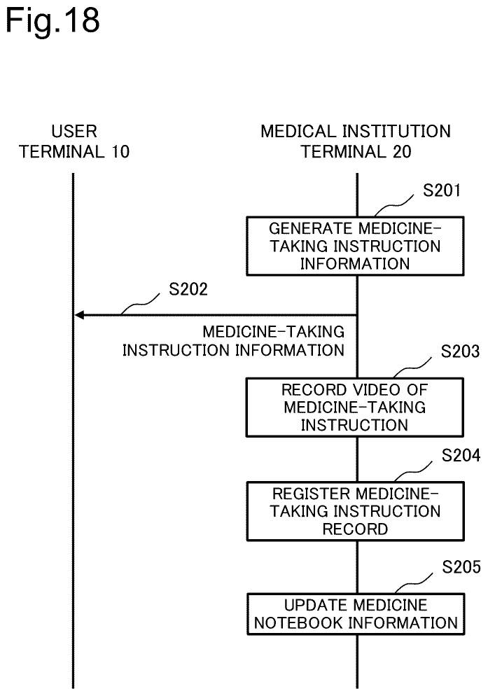

Next, the medicine-taking instruction process (step S20) of the medical institution terminal 20 illustrated in FIG. 14 will be described. FIG. 18 is a sequence diagram illustrating an example of the medicine-taking instruction process.

After the confirmation process of the patient and the attendant is completed, the medicine-taking instruction processing unit 203 of the medical institution terminal 20 generates a medicine-taking instruction ID, and generates the record of the medicine-taking instruction information 214 in the medical DB 21 using the patient ID and the attendant ID transmitted from the confirmation unit 201 (step S201). Further, the medicine-taking instruction processing unit 203 adds a medicine prescribed by the doctor to the patient and information (prescription information) regarding the prescription to the medicine-taking instruction information 214.

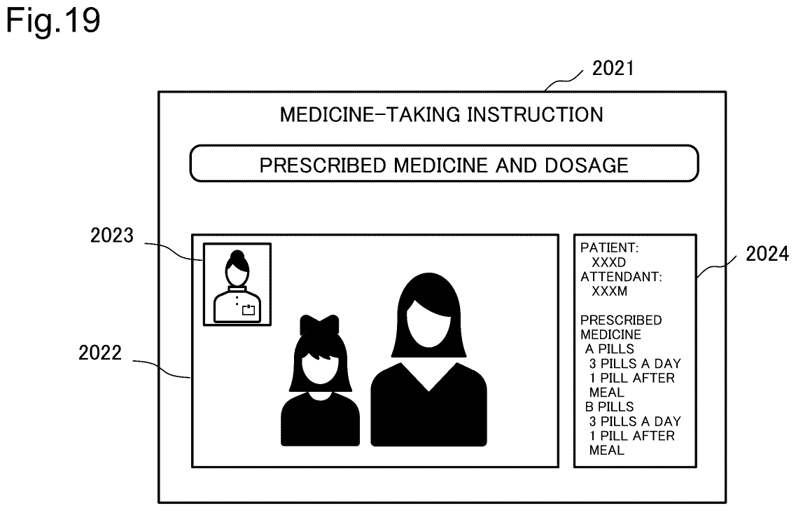

FIG. 19 is a diagram illustrating an example of screen output of the medical institution terminal 20. In the display 2024 illustrated in FIG. 19, information on the prescribed medicine of the patient is displayed based on the information on the medicine-taking instruction information 214. The doctor or pharmacist looks at the display 2024 and explains the medicine prescribed for the patient and the dosage thereof to the patient and the attendant. The medicine-taking instruction processing unit 203 transmits a part of the medicine-taking instruction information 214 to the user terminal 10 (step S202).

While the video and audio data of the patient, the attendant, the doctor, or the pharmacist are transmitted and received between the user terminal 10 and the medical institution terminal 20, the medicine-taking instruction processing unit 203 records the video and audio data of the medicine-taking instruction (step S203).

In the medicine-taking instruction, a pharmacist and a doctor listen to symptoms from a patient and an attendant, and then explain the effect of a prescribed medicine, a method of taking the medicine, a possible side effect, a response to forgetting to take the medicine, and a storage method. The pharmacist and the doctor confirm and answer questions from the patient and the attendant.

After the medicine-taking instruction, the medicine-taking instruction processing unit 203 registers the medicine history of the patient in the medicine-taking instruction record of the medicine-taking instruction information 214 (step S204). The medicine-taking instruction record also includes video and audio data from the start to the end of the medicine-taking instruction. The medicine-taking instruction processing unit 203 registers the medicine-taking information of the medicine-taking instruction information 214 in the medicine notebook information after the completion of the medicine-taking instruction. In the medicine notebook information, medicine-taking information for each medicine-taking instruction is accumulated.

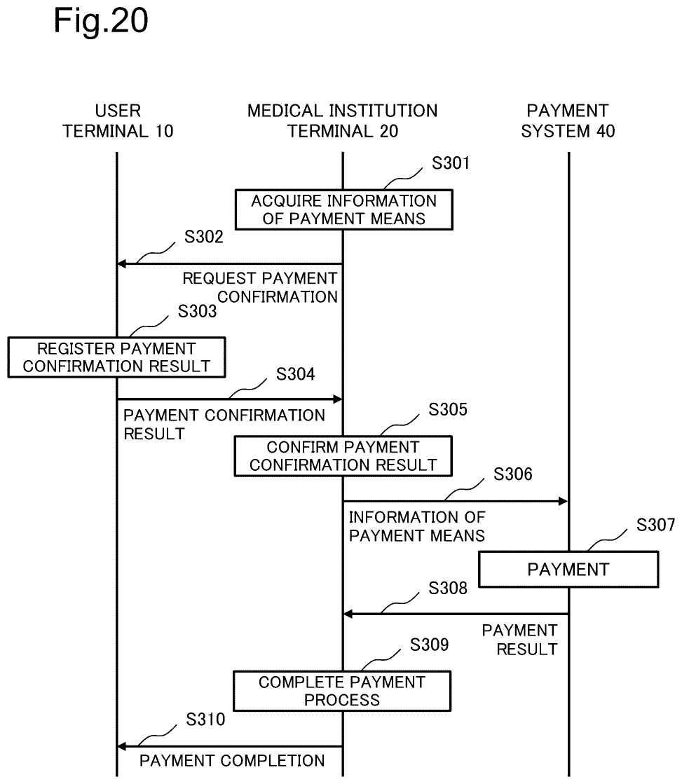

Next, the payment process (step S30) of the medical institution terminal 20 illustrated in FIG. 14 will be described. FIG. 20 is a sequence diagram illustrating an example of the payment process.

The payment processing unit 204 of the medical institution terminal 20 refers to the payment means information 215 in the medical DB 21, and acquires a payment means associated with the patient or the attendant (step S301). The payment processing unit 204 transmits a payment confirmation request for confirming a payment means associated with the patient or the attendant to the patient or the attendant who is the payer in the accounting of the online medicine-taking instruction (step S302). The payment confirmation request includes the name of the payer and information on the payment means. The information of the payment means is, for example, card information.

When the user terminal 10 receives the payment confirmation request, the processing unit 101 displays the contents of the payment confirmation request on the screen, receives the confirmation result, and registers the payment confirmation result (step S303). The confirmation result is, for example, payment approval or payment rejection. The payment approval is information indicating that the payer accepts the payment by the payment means. The user terminal 10 transmits the registered payment confirmation result to the medical institution terminal 20 (step S304). The payment processing unit 204 confirms the payment confirmation result (step S305), and when the payment confirmation result indicates payment approval, transmits information on the payment means to the payment system 40 (step S306), and requests payment. The payment system 40 makes a payment with the transmitted information on the payment means (step S307), and transmits a payment result to the medical institution terminal 20 (step S308).

The payment processing unit 204 receives the payment result from the payment system 40, and when the payment result indicates payment completion, the payment process is completed (step S309). The medical institution terminal 20 transmits payment completion information to the user terminal 10 (step S310).

The payment processing unit 204 may perform the age estimation process based on the face images of the patient and the attendant when specifying the payer and acquiring the information on the payment means in step S301. As a result of the age estimation process, the payment processing unit 204 specifies a patient or an attendant with a face image with a high estimated age as a payer. The payment processing unit 204 transmits a payment confirmation request for confirming a payment means associated with the specified patient or attendant to the user terminal 10. The payment confirmation request is, for example, “Is the payer Mr./Ms. ∘∘?”.

When the processing unit 101 of the user terminal 10 outputs the payment confirmation display, the patient or the attendant can confirm and register the payer and the payment means.

Next, the medicine-taking management (step S40) of the medical institution terminal 20 illustrated in FIG. 14 will be described. The medicine-taking management is a process of managing medicine-taking of a patient. The medicine-taking management includes a medicine-taking notification process and medicine-taking confirmation process.

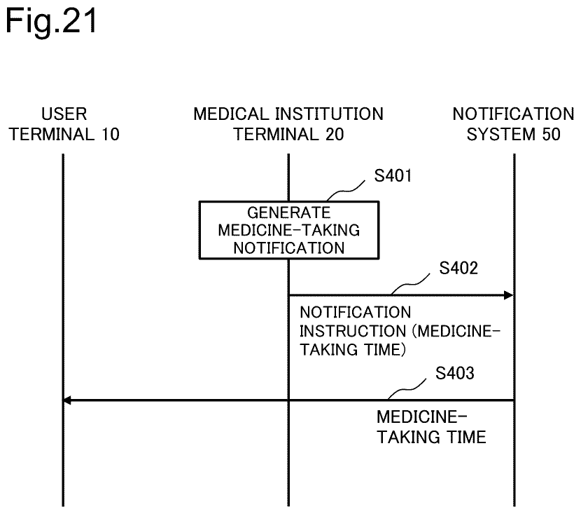

FIG. 21 is a sequence diagram illustrating an example of an operation related to the medicine-taking notification process of the medicine-taking management. The medicine-taking notification is notified to the user terminal 10 used by the patient or the attendant via the notification system 50.

The medicine-taking management unit 205 of the medical institution terminal 20 refers to the medicine-taking information and the medicine-taking instruction record in the medical DB 21, acquires a time zone when the patient takes the medicine, and generates a medicine-taking notification (step S401). The notification processing unit 206 of the medical institution terminal 20 generates a notification instruction for notifying the patient and the attendant, and transmits the notification instruction including the medicine-taking notification to the notification system 50 (step S402). The notification system 50 receives the notification instruction and notifies the e-mail addresses of the patient and the attendant of the medicine-taking notification based on the notification instruction (step S403).

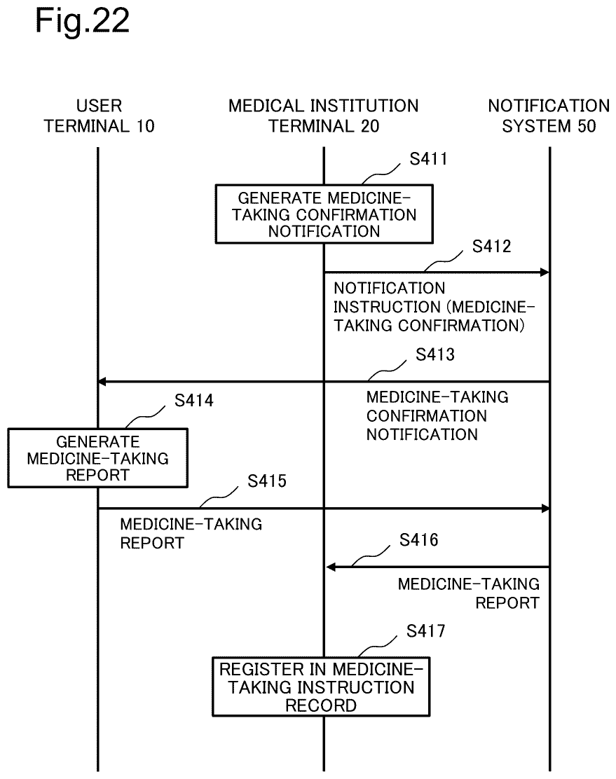

FIG. 22 is a sequence diagram illustrating an example of the operation of the medicine-taking confirmation process of the medicine-taking management. Hereinafter, the medicine-taking confirmation process via the notification system 50 will be described.

The medical institution terminal 20 sends the medicine-taking confirmation notification to the user terminal 10, receives the medicine-taking report from the user terminal 10, and confirms the medicine-taking status of the patient.

Specifically, the medicine-taking management unit 205 refers to the medicine-taking information and the medicine-taking instruction record in the medical DB 21, and generates a medicine-taking confirmation notification of the patient (step S411). The notification processing unit 206 generates a notification instruction for notifying the patient and the attendant, and transmits the notification instruction including the medicine-taking confirmation notification to the notification system 50 (step S412). The notification system 50 receives the notification instruction and notifies the e-mail addresses of the patient and the attendant of the medicine-taking confirmation notification based on the notification instruction (step S413). For example, the notification to the patient and the attendant may be a short message or an application used in an SNS.

The processing unit 101 of the user terminal 10 generates a medicine-taking report (step S414). The medicine-taking report includes information indicating that the patient has taken the medicine according to the medicine-taking instruction. For example, the medicine-taking report may be a video obtained by capturing an image of the patient taking the medicine. The processing unit 101 returns the medicine-taking report to the transmission source of the medicine-taking confirmation notification (step S415). The notification system 50 transmits the medicine-taking report from the user terminal 10 to the medical institution terminal 20 (step S416).

The medicine-taking management unit 205 registers the medicine-taking status of the patient in the medicine-taking instruction record of the medical DB 21 based on the medicine-taking report (step S417).

The medicine-taking management unit 205 may generate a remaining medicine notification of a medicine prescribed with reference to the prescription instruction record of the patient updated by the medicine-taking report of the patient. The remaining medicine notification may include a date when the number of remaining medicines of the patient becomes 0 when the medicine is taken according to the prescription. The remaining medicine notification is notified to the user terminal 10 via the notification system 50.

### Effects of First Example Embodiment

According to the first example embodiment, the convenience of the system in the online medicine-taking instruction is improved. Specifically, in a case where a plurality of persons receive online medicine-taking instruction such as a patient and an attendant, the medical institution terminal 20 can confirm the relationship of the person shown on the screen on behalf of the medical worker. This is because the confirmation unit 201 confirms whether the first face image and the second face image included in the image obtained by capturing the persons who receive the online medicine-taking instruction correspond to the relationship between the patient and the attendant, and the output unit 202 outputs the confirmation result.

### Second Example Embodiment

A medical institution terminal according to a second example embodiment will be described with reference to the drawings. The medical institution terminal 20 according to the second example embodiment is also communicably connected to the user terminal 10 and the authentication system 30 via the network 60 similarly to the medical institution terminal 20 according to the first example embodiment. The medical institution terminal 20 is communicably connected to the medical DB 21.

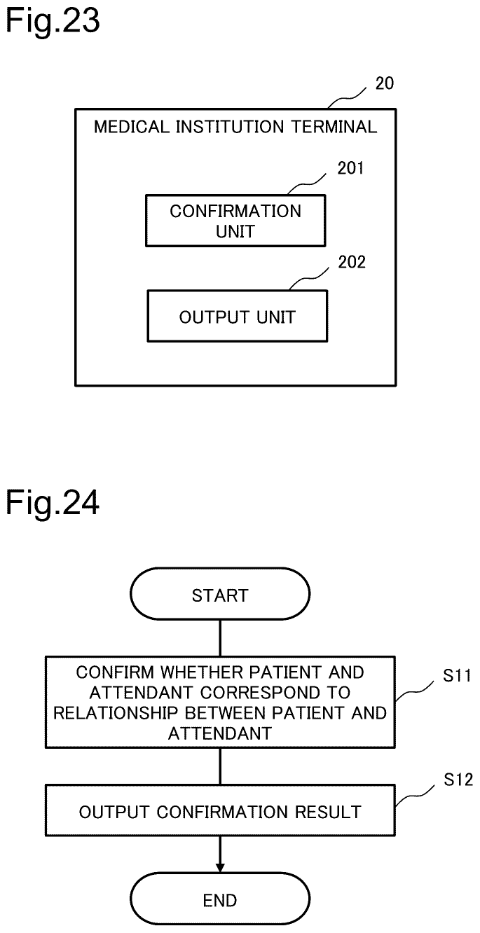

FIG. 23 is a block diagram illustrating an example of a configuration of the medical institution terminal 20. The medical institution terminal 20 includes a confirmation unit 201 and an output unit 202. The configuration of the medical institution terminal 20 according to the second example embodiment is a configuration in which the medicine-taking instruction processing unit 203, the payment processing unit 204, the medicine-taking management unit 205, and the notification processing unit 206 are omitted from the medical institution terminal 20 according to the first example embodiment. The medical institution terminal is also referred to as an information processing apparatus. The medical institution terminal 20 includes a processor and a memory, and the processor executes a processing program stored in the memory to implement various functions including the functions of the confirmation unit 201 and the output unit 202.

The medical institution terminal 20 receives an image obtained by capturing an image of the patient who receives the online medicine-taking instruction and the attendant from the user terminal 10. The confirmation unit 201 confirms whether the first face image and the second face image included in the image obtained by capturing an image of the persons who receive the online medicine-taking instruction correspond to the relationship between the patient and the attendant. Details will be described below.

The confirmation unit 201 transmits the acquired face image to the authentication system 30 and collates the face image with the face information registered in the authentication system 30. When the face information of the face image matches the face information of the authentication system 30, the authentication system 30 transmits the person ID relevant to the registered face information to the medical institution terminal 20 as the collation result. When the person ID of the collation result is received, the confirmation unit 201 refers to the human identification information 213 of the medical DB 21 and extracts a patient ID or an attendant ID that matches the person ID of the collation result.

The confirmation unit 201 confirms whether the extracted patient ID and attendant ID correspond to a relationship between the patient ID and the attendant ID registered in the patient information 211 of the medical DB 21. In a case where the relationship corresponds to the relationship between the patient and the attendant registered in the patient information 211, the confirmation unit 201 sends the confirmation result to the output unit 202. The confirmation result may include the patient ID of the patient whose identity verification has been completed and the attendant ID of the attendant. The confirmation result may include a patient name, an attendant name, or a relationship with a patient.

The output unit 202 outputs the confirmation result. The output unit 202 may be a display of information indicating that the relationship corresponds to the relationship between the patient and the attendant. For example, in a case where the face images of the patient and the attendant are displayed on the same screen, a mark surrounding the faces may be blinked at the same time, or a mark surrounding both the faces together may be displayed. The information indicating that the relationship corresponds to the relationship between the patient and the attendant may be a text or the like. The output unit 202 may display and output the person who receives the medicine-taking instruction, for example, the patient name and the attendant name on the monitor screen.

The medical institution terminal 20 according to the second example embodiment may include the medical DB 21 or the authentication system 30.

FIG. 24 is a flowchart illustrating an example of an operation of the medical institution terminal 20 according to the second example embodiment. The user terminal 10 and the medical institution terminal are communicably connected to each other to be able to transmit and receive various types of information.

The user terminal 10 transmits, to the medical institution terminal 20, captured data including the face of the patient receiving the online medicine-taking instruction and the attendant. When the medical institution terminal 20 receives the captured data of the patient and the attendant, the confirmation unit 201 acquires a face image (first face image) of the patient and a face image (second face image) of the attendant. The medical institution terminal 20 transmits the face images of the patient and the attendant to the authentication system 30. The authentication system 30 collates the received face image of the patient (first face image) and the received face image of the attendant (second face image) with face information registered in advance, and transmits a collation result to the medical institution terminal 20. In a case where the face information of the patient and the face information of the attendant coincide, the collation result including the person ID relevant to the face information registered in the authentication system 30 is transmitted.

When the person ID is received as the collation result, the confirmation unit 201 refers to the human identification information 213 of the medical DB 21 and extracts the patient ID or the attendant ID that matches the person ID of the collation result. The confirmation unit 201 confirms whether the extracted patient ID and attendant ID correspond to a relationship between the patient ID and the attendant ID registered in the patient information 211 of the medical DB 21 (step S11). As a result of the confirmation, in a case where the relationship corresponds to the relationship between the patient and the attendant, the confirmation unit 201 sends the confirmation result to the output unit 202. The output unit 202 outputs the confirmation result (step S12). The output of the confirmation result is display of a mark indicating that the relationship corresponds to the relationship between the patient and the attendant. By changing the color, shape, and the like of the marks of the patient and the attendant, it may be possible to distinguish between the patient and the attendant. The output unit 202 may display and output the names of the patient and the attendant.

### Effects of Second Example Embodiment

According to the medical institution terminal 20 of the second example embodiment, the convenience of the system in the online medicine-taking instruction can be improved. Specifically, in a case where a plurality of persons receive online medicine-taking instruction such as a patient and an attendant, the medical institution terminal 20 can confirm the relationship of the person shown on the screen on behalf of the medical worker. This is because the confirmation unit 201 confirms whether the first face image and the second face image included in the image obtained by capturing the persons who receive the online medicine-taking instruction correspond to the relationship between the patient and the attendant, and the output unit 202 outputs the confirmation result.

(Hardware Configuration)

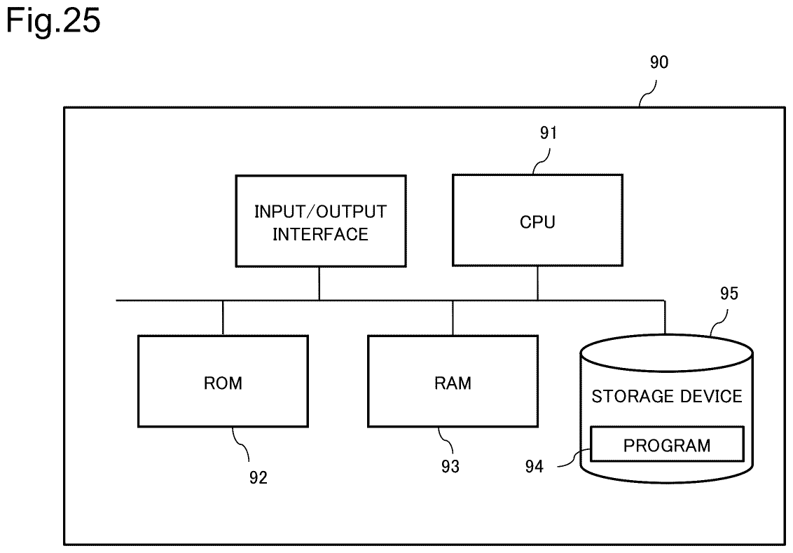

FIG. 25 is a diagram illustrating an example of a hardware configuration of a computer. The user terminal 10, the medical institution terminal 20, the authentication system 30, the payment system 40, and the notification system 50 described above are achieved by executing a program (software program, computer program) in the CPU 91 of the computer 90 illustrated in FIG. 25. Specifically, the functions of the components of the medical institution terminal 20 are implemented by executing a program. These configurations may be achieved by a central processing unit (CPU) 91 reading a program 94 from a read only memory (ROM) 92 or a storage device 95 and executing the read program 94 using the CPU 91 and a random access memory (RAM) 93. The present disclosure described using the above-described example embodiment as an example can be regarded as being configured by a computer readable recording medium in which a code representing a computer program or a code representing the computer program is stored. The computer-readable recording medium is, for example, a storage device 95, a removable magnetic disk medium (not illustrated), an optical disk medium, a memory card, or the like. The components of each example embodiment may be dedicated hardware by an integrated circuit.

The present disclosure is not limited to the above-described example embodiments, and various modifications can be made, and example embodiments obtained by appropriately combining configurations, operations, and processes disclosed in different example embodiments are also included in the technical scope of the present disclosure.

The present disclosure is not limited to the above-described example embodiments. That is, the present invention can apply various aspects that can be understood by those of ordinary skill in the art without departing from the spirit and scope of the present disclosure.

[Supplementary Note 1]

An information processing apparatus including:
a confirmation means configured to confirm whether a first face image and a second face image included in an image obtained by capturing persons who receive online medicine-taking instruction correspond to a relationship between a patient and an attendant; and an output means configured to output a confirmation result.

[Supplementary Note 2]

The information processing apparatus according to Supplementary Note 1, in which the first face image and the second face image are included in a same image.

[Supplementary Note 3]

The information processing apparatus according to Supplementary Note 1 or 2, further including:
a storage means configured to store patient information indicating a relationship between the patient and the attendant.

[Supplementary Note 4]

The information processing apparatus according to any one of Supplementary Notes 1 to 3, in which
when the relationship is satisfied,
the output means outputs a display indicating identification of the patient or the attendant with respect to the first face image and the second face image.

[Supplementary Note 5]

The information processing apparatus according to any one of Supplementary Notes 1 to 4, in which
when the relationship is satisfied,
the output means outputs a display indicating a name of the patient and a name of the attendant.

[Supplementary Note 6]

The information processing apparatus according to any one of Supplementary Notes 1 to 3, in which
when the relationship is not satisfied, the output means outputs error information indicating that there is no relationship between the persons of the first face image and the second face image.

[Supplementary Note 7]

The information processing apparatus according to any one of Supplementary Notes 1 to 6, further including:
a medicine-taking instruction processing means configured to register medicine-taking instruction information of the online medicine-taking instruction in medicine notebook information of the patient.

[Supplementary Note 8]

The information processing apparatus according to Supplementary Note 7, in which
the medicine-taking instruction information includes a medicine-taking instruction date, a prescription, a patient name, and an attendant name.

[Supplementary Note 9]

The information processing apparatus according to any one of Supplementary Notes 1 to 8, further including:
a payment processing means configured to settle accounting for the online medicine-taking instruction by referring to payment means information indicating payment means associated with the patient or the attendant.

[Supplementary Note 10]

The information processing apparatus according to Supplementary Note 9, in which
the confirmation means executes an age estimation process using the face images of the patient and the attendant, and
the payment processing means sets one of the patient and the attendant having a higher estimated age as a payer of the accounting.

[Supplementary Note 11]

The information processing apparatus according to any one of Supplementary Notes 1 to 10, further including:
a medicine-taking management means configured to notify a user terminal of the patient or the attendant of a medicine-taking timing of the patient in a time zone in which the patient takes a medicine.

[Supplementary Note 12]

The information processing apparatus according to Supplementary Note 11, in which
the medicine-taking management means confirms a medicine-taking status of the patient based on a medicine-taking report from a user terminal of the patient or the attendant.

[Supplementary Note 13]

The information processing apparatus according to Supplementary Note 12, in which
the medicine-taking report includes a video obtained by capturing an image of the patient taking a medicine.

[Supplementary Note 14]

An information processing method including:
confirming whether a first face image and a second face image obtained by capturing an image of a person who receives an online medicine-taking instruction correspond to a relationship between a patient and an attendant; and
outputting a confirmation result.

[Supplementary Note 15]

A recording medium storing a program for causing a computer to execute:
confirming whether a first face image and a second face image obtained by capturing an image of a person who receives an online medicine-taking instruction correspond to a relationship between a patient and an attendant; and
outputting a confirmation result.

### REFERENCE SIGNS LIST

10 user terminal
20 medical institution terminal
21 medical DB
30 authentication system
40 payment system
50 notification system
201 confirmation unit
202 output unit
203 medicine-taking instruction processing unit
204 payment processing unit
205 medicine-taking management unit
206 notification processing unit

What is claimed is:

1. An information processing apparatus comprising:
one or more memories storing instructions; and
one or more processors configured to execute the instructions to:
confirm whether a first face image and a second face image included in an image obtained by capturing persons who receive online medicine-taking instruction correspond to a relationship between a patient and an attendant; and
output a confirmation result.

2. The information processing apparatus according to claim 1, wherein
the first face image and the second face image are included in a same image.

3. The information processing apparatus according to claim 1, wherein the one or more memories
store patient information indicating a relationship between the patient and the attendant.

4. The information processing apparatus according to claim 1, wherein the one or more processors are configured to execute the instructions to:
when the relationship is satisfied,
output a display indicating identification of the patient or the attendant with respect to the first face image and the second face image.

5. The information processing apparatus according to claim 1, wherein the one or more processors are configured to execute the instructions to:
when the relationship is satisfied,
output a display indicating a name of the patient and a name of the attendant.

6. The information processing apparatus according to claim 1, wherein the one or more processors are configured to execute the instructions to:
when the relationship is not satisfied, output error information indicating that there is no relationship between the persons of the first face image and the second face image.

7. The information processing apparatus according to claim 1, wherein the one or more processors are configured to execute the instructions to:
register medicine-taking instruction information of the online medicine-taking instruction in medicine notebook information of the patient.

8. The information processing apparatus according to claim 7, wherein
the medicine-taking instruction information includes a medicine-taking instruction date, a prescription, a patient name, and an attendant name.

9. The information processing apparatus according to claim 1, 1 wherein the one or more processors are configured to execute the instructions to:
settle accounting for the online medicine-taking instruction by referring to payment means information indicating payment means associated with the patient or the attendant.

10. The information processing apparatus according to claim 9, wherein the one or more processors are configured to execute the instructions to:
execute an age estimation process using a face image of the patient and the attendant, and
set one of the patient and the attendant having a higher estimated age as a payer of the accounting.

11. The information processing apparatus according to claim 1, wherein the one or more processors are configured to execute the instructions to:
notify a user terminal of the patient or the attendant of a medicine-taking timing of the patient in a time zone in which the patient takes a medicine.

12. The information processing apparatus according to claim 11, wherein the one or more processors are configured to execute the instructions to:
confirm a medicine-taking status of the patient based on a medicine-taking report from a user terminal of the patient or the attendant.

13. The information processing apparatus according to claim 12, wherein the medicine-taking report includes a video obtained by capturing an image of the patient taking a medicine.

14. An information processing method comprising:
confirming whether a first face image and a second face image obtained by capturing an image of a person who receives an online medicine-taking instruction correspond to a relationship between a patient and an attendant; and
outputting a confirmation result.

15. A recording medium storing a program for causing a computer to execute:
confirming whether a first face image and a second face image obtained by capturing an image of a person who receives an online medicine-taking instruction correspond to a relationship between a patient and an attendant; and
outputting a confirmation result.
